(19) United States
(12) Patent Application Publication
(10) Pub. No.: US 2007/0004032 A1
(43) Pub. Date: Jan. 4, 2007

(54) Chip device for a thermally cycled reaction

(75) Inventors: Gwo-Bin Lee, Tainan City (TW); Chih-Hao Wang, Taipei Hsien (TW); Chia-Sheng Liao, Taipei City (TW)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: National Cheng Kung University, Tainan City (TW)

(21) Appl. No.: 11/316,576

(22) Filed: Dec. 21, 2005

(30) Foreign Application Priority Data
Jun. 29, 2005 (TW) 094121783

(51) Int. Cl. C12M 1/34

(52) U.S. Cl. 435/288.5; 435/303.1

(57) ABSTRACT

A chip device for a thermally cycled reaction includes a looped pressure channel divided into a plurality of angularly oriented sub-channels, and a looped fluid channel substantially corresponding to and disposed below the looped pressure channel. The fluid channel has a plurality of temperature zones below the respective sub-channels. A looped membrane is disposed between the fluid channel and the pressure channel. The membrane is inflated or deflated by the pressure in the sub-channels so as to block or unblock the fluid channel partially or completely. A fluid sample is movable by the membrane to an unblocked part of the fluid channel from a blocked part of the fluid channel so that the fluid sample may be circulated repeatedly along the fluid channel.

### CROSS-REFERENCE TO RELATED APPLICATION

This application claims priority from Taiwanese Utility Application No. 94121783, filed on Jun. 29, 2005.

### BACKGROUND OF THE INVENTION

1. Field of the Invention

This invention relates to a chip device, more particularly to a chip device for a thermally cycled reaction, such as, a Polymerase Chain Reaction (PCR).

2. Description of the Related Art

As micro electro mechanical system technology becomes mature, developments thereof have been made in many different fields, especially in the miniaturization of biomedical detection instruments, that has played an important role to stimulate development. Micro fluidic biomedical detector chips employing the micro electro mechanical system technology provide advantages, such as high detection performance, disposability, portability, low sample and specimen consumption, low energy consumption, reduced size, low cost, etc. Therefore, integration of related fluidic systems of a cycled reaction in a single chip has the highest development potential and marketing value.

Polymerase Chain Reaction (PCR) is an important technique for amplification of a DNA sequence and includes denaturation, annealing, and extension of a target DNA sequence under controlled temperatures. While there are many reports relating to researches on PCR chips, only two types of PCR chips have been used commonly for polymerase chain reaction (PCR) assays since 1933; one being a sample-moving type in which a sample is moved between several regions which are heated to different controlled temperatures, the other being a temperature-cycling type in which a sample is placed within a fixed region whose temperature is changed within a cycled temperature.

Referring to FIG. 1, there is shown a typical apparatus for a thermally cycled polymerase chain reaction which includes a microchip 100 having a base 10 provided with a heating device 101 and a temperature sensor 103 in a particular region. The heating device 101 and the temperature sensor 103 are formed of thin metal films which are produced through lithography and metal deposition techniques. The metal films extend to the periphery of the base 10 and are connected electrically to a power source and an instrument (not shown). A thin glass plate 13 covers the heating device 101 and the temperature sensor 103 and has a receiving chamber 15 which is formed using polydimethylsiloxane (PDMS).

In operation, a fluid sample for PCR is placed in the receiving chamber 15, and the receiving chamber 15 and the fluid sample is heated by the heating device 101 through automatic control. The heating temperature is therefore increased and decreased repeatedly within a temperature cycle.

In the microchip 100, although no drive unit is needed to drive the fluid sample, it takes time for the heating device 101 to change between upper and lower temperatures. In addition, it is difficult to control the temperature to stay accurately at a predetermined level required for the PCR reaction. In case the temperature is inaccurate in the course of a reaction step, a significant change will occur in the entire reaction process so that no correct result can be obtained.

Referring to FIG. 2, there is shown another conventional PCR apparatus 200 which has a larger size compared to the microchip 100. The apparatus 200 includes three heating devices 21 which are provided respectively with three different preset temperatures, and a fluid bearing layer 22 disposed on the heating devices 21. The preset temperatures of the heating devices 21 may be 53 deg., 72 deg., and 95 deg., respectively; the 53 deg. temperature may be changed depending on the different types of DNA. The fluid bearing layer 22 is provided with a microchannel system 221 which is densely distributed in the fluid bearing layer 22 and which is heated by the three heating devices 21 to provide different temperature zones.

In use, a fluid sample is introduced into the microchannel system 221 and is thereafter driven by a drive unit, such as, a fluid pump (not shown), to flow through the different temperature zones of the microchannel system 221. The temperature of the fluid sample is cycled when the fluid sample flows through the temperature zones.

However, the PCR apparatus 200 is disadvantageous due to the complicated construction of the microchannel system 221. On the other hand, the fluid sample has to be carefully controlled at a constant rate in the microchannel system 22 because the time required by the fluid sample to flow through each temperature zone should be enough so that the corresponding reaction step can be carried out.

In addition, the length of the microchannel system 221 in each temperature zone is fixed and cannot be altered. Therefore, the reaction time in each temperature zone and the number of PCR cycles that can be run in the apparatus 200 are also fixed. The numbers provided in FIG. 2 indicate the points where the designated numbers of cycles are completed, and a reaction product can be taken out from the PCR apparatus 200 only at one of the designated points. It is difficult to alter the number of cycles and/or the reaction time designed in the apparatus 200.

### SUMMARY OF THE INVENTION

An object of the present invention is to provide a chip device which overcomes the disadvantages encountered with the aforesaid prior art.

Another object of the present invention is to provide a chip device which is simple in construction and operation.

According to the present invention, a chip device for a thermally cycled reaction includes a looped pressure channel divided into a plurality of angularly oriented sub-channels, and a looped fluid channel substantially corresponding to and disposed below the looped pressure channel. The fluid channel has a plurality of temperature zones disposed below the sub-channels, respectively. A membrane is disposed between the fluid channel and the pressure channel. The membrane is inflated or deflated by the pressure in the sub-channels so as to block or unblock the fluid channel partially or completely. When the membrane is deflated partially, one of the temperature zones is unblocked and the remaining of the temperature zones are blocked so that a fluid sample introduced into the fluid channel may be moved to the unblock one of the temperature zones from the remaining temperature zones.

### BRIEF DESCRIPTION OF THE DRAWINGS

Other features and advantages of the present invention will become apparent in the following detailed description of the preferred embodiment of the invention, with reference to the accompanying drawings, in which:

FIG. 1 is a perspective view of a conventional chip apparatus;

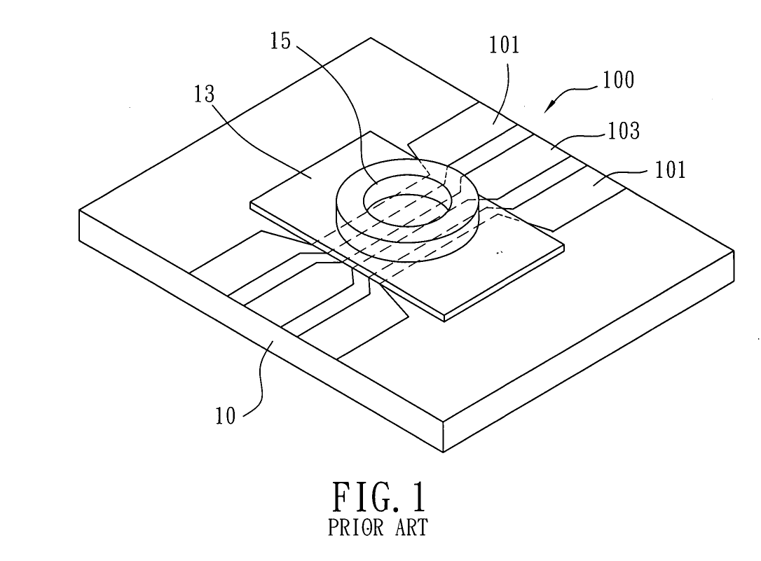

FIG. 2 is a schematic view of another conventional chip apparatus;

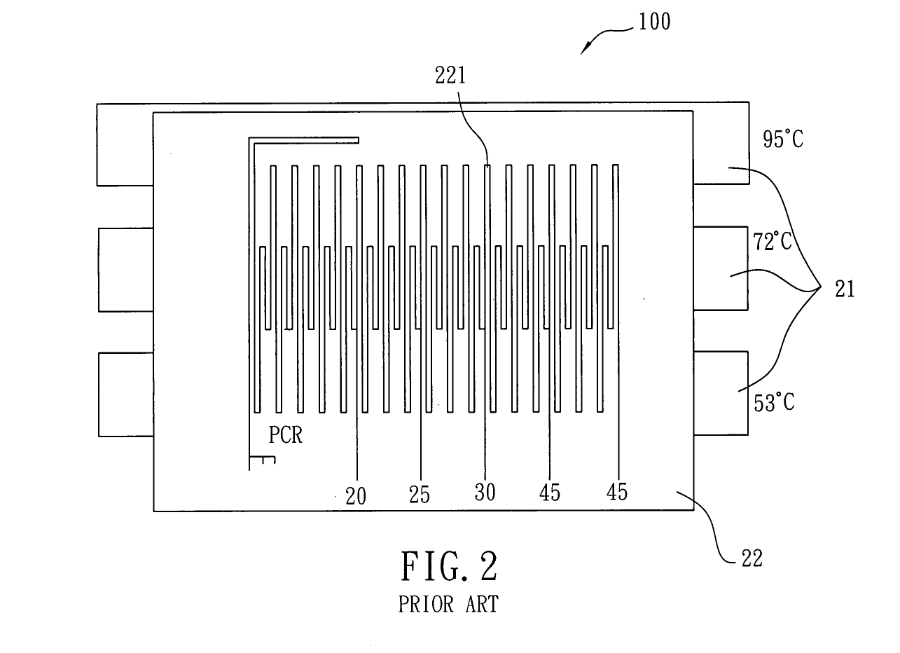

FIG. 3 is an exploded view of a chip device embodying the present invention;

FIG. 4 is a schematic view showing three heating devices and three temperature sensors used in the chip device;

FIG. 5 is a schematic view showing three temperature sensors used in the chip device;

FIG. 6 is a plan view of the chip device;

FIG. 7 is a sectional view taken along line 7-7 of FIG. 6; and

FIG. 8 is a sectional view taken along line 8-8 of FIG. 6.

### DETAILED DESCRIPTION OF THE PREFERRED EMBODIMENT

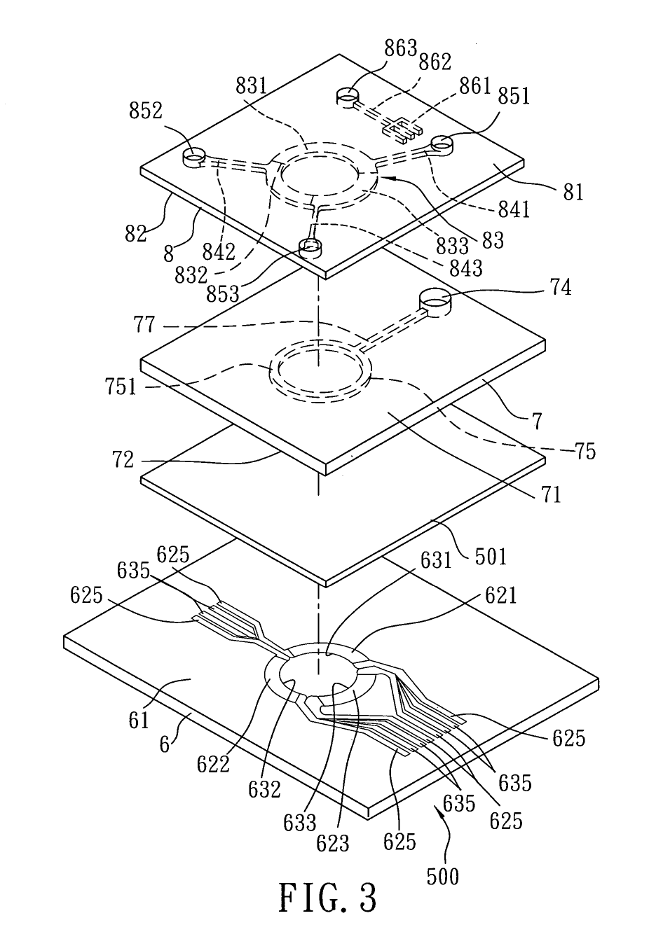

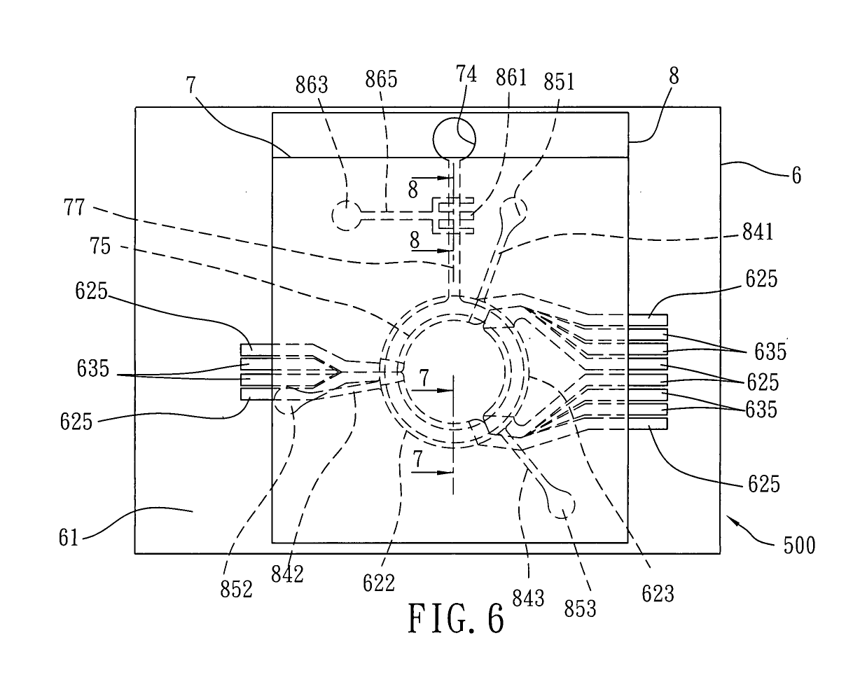

Referring to FIGS. 3 and 6, a chip device 500 according to the first preferred embodiment of the present invention includes a substrate 6, a fluid bearing layer 7, and a pressure controlled layer 8.

The substrate 6 in this embodiment is a glass plate 6 having a top surface 61 provided with three heating devices, i.e. first, second and third heating devices 621, 622, 623 each of which has a film of an arcuate shape formed by microlithography and metal vaporizing techniques and which are oriented angularly or annularly. Each film of the first, second and third heating devices 621, 622, 623 has a length substantially equal to one third of a circumferential length of an annulus and is spaced from the other films by micro gaps. Each of the first, second and third heating devices 621, 622 and 623 is connected to two conductor films 625 provided on the top surface 61 and extending to two opposite lateral sides thereof.

The substrate 6 further includes first, second and third temperature sensors 631, 632 and 633 each having a film also formed by microlithography and metal vaporizing techniques. The films of the first, second and third temperature sensors 631, 632 and 633 are also oriented angularly or annularly and each have a length substantially equal to one third of a circumferential length of an annulus. Each film of the first, second and third temperature sensors 631, 632, 633 extends along an inner edge of the corresponding film of the first, second or third heating device 621, 622 or 623 and has two ends respectively connected to two conductor films 635 which extend to the lateral sides of the top surface 61 substantially in a parallel relationship with the conductor films 625 of the heating devices 621, 622, 623.

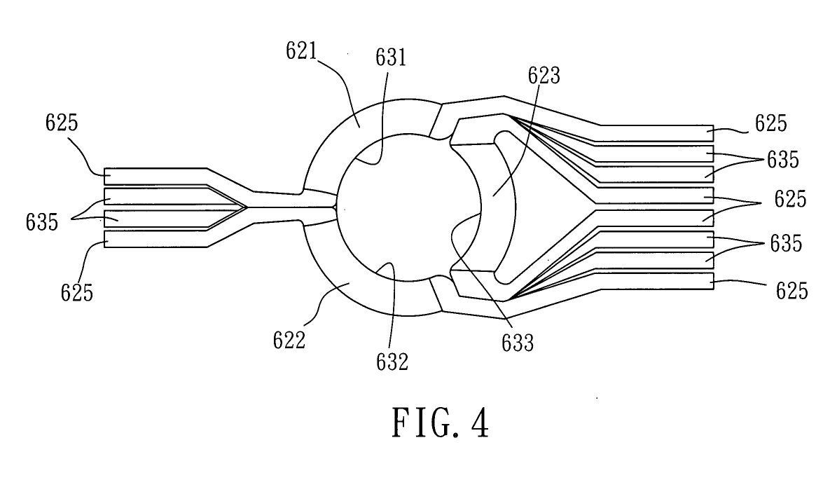

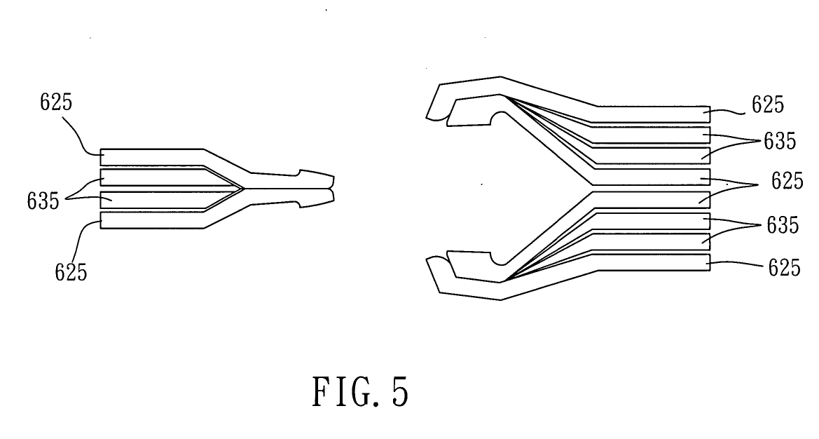

Referring to FIGS. 4 and 5, the first, second and third heating devices 621, 622, 623 and the first, second and third temperature sensors 631, 632, 633 are made of platinum (Pt), whereas the conductor films 625, 635 thereof are made of gold. The platinum is deposited on the top surface 61 prior to the deposition of the gold. Gold is used advantageously therein because of its small resistance and good electrical conduction, and platinum is used for its high resistance and good heating properties.

Referring once again to FIG. 3, the fluid bearing layer 7 is a transparent thin film layer which is made of polydimethylsiloxane. The fluid bearing layer 7 includes a top surface 71, a bottom surface 72, a receptacle 74 extending through the top and bottom surfaces 71, 72, a circularly looped fluid channel 75, and a micro-channel 77. The top surface 71 is recessed to form the fluid channel 75 and the micro-channel 77. The fluid channel 75 substantially corresponds to the annulus of the first, second and third heating devices 621, 622, 623. The micro-channel 77 is used to interconnect the receptacle 74 and the fluid channel 75.

The pressure controlled layer 8 is also made of polydimethylsiloxane and includes a top surface 81, a bottom surface 82, and a circularly looped pressure channel 83 substantially corresponding to the looped fluid channel 75. The bottom surface 82 is further recessed to form the pressure channel 83. The pressure channel 83 is divided into three angularly oriented non-communicating arcuate sub-channels 831, 832, 833. The bottom surface 82 is further recessed to form three micro-passages 841, 842 and 843 which are connected fluidly to the sub-channels 831, 832, 833, respectively. Three pressure inlet/outlet holes 851, 852 and 853 extend through the top and bottom surfaces 81 and 82 and are connected fluidly to the ends of the micro-passages 841, 842, 843, respectively.

The bottom surface 82 of the pressure controlled layer 8 is further recessed to form a substantially E-shaped pressure slot 861 and a micro-passage 865, both of which do not extend through the top surface 81. The micro-passage 865 connects fluidly the E-shaped pressure slot 861 with another pressure inlet/outlet hole 863 that extends through the top and bottom surfaces 81 and 82.

In assembly, the pressure controlled layer 8 is superimposed over the fluid bearing layer 7, as shown in FIG. 6. The substrate 6 is disposed below the fluid bearing layer 7 with the heating devices 621, 622, 623 being aligned with the fluid channel 75. A glass partition 501 is disposed between the substrate 6 and the fluid bearing layer 7 so that the partition 501 is interposed between the heating devices 621, 622, 623 and the fluid channel 75. The fluid channels 75 will have three different temperature zones when heated by the heating devices 621, 622, 623 which are preset at three different temperatures during the operation of the chip device 500.

The conductor films 625, 635 are exposed at the lateral sides of the substrate 6 and are connected to an external power source and an external instrument (not shown). The receptacle 74 in the fluid bearing layer 7 has one end covered by the partition 501 and serves to receive a fluid sample.

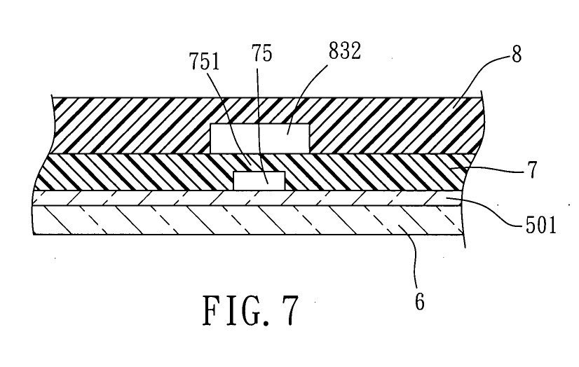

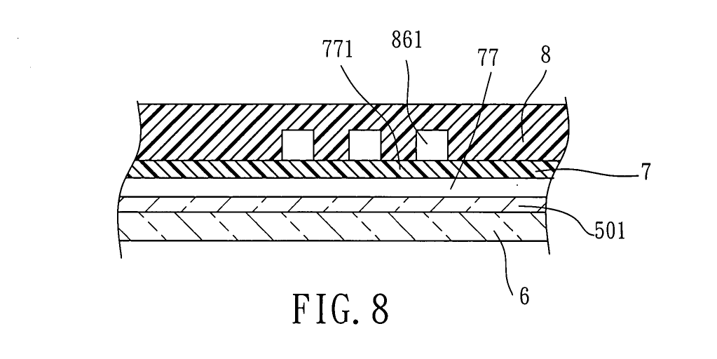

The bottom surface 82 of the pressure controlled layer 82 contacts the top surface 71 of the fluid bearing layer 7. The pressure channel 83 and the E-shaped pressure slot 861 are superimposed over the fluid channel 75 and the micro-channel 77, respectively. As the fluid channel 75 and the micro-channel 77 do not extend through the top surface 71 of the fluid bearing layer 7, the fluid bearing layer 7 has an annular portion between the fluid channel 75 and the pressure channel 83, which defines a looped or annular membrane 751 (as best shown in FIG. 7), and a portion between the micro-channel 77 and the E-shaped pressure slot 861, which defines a membrane valve 771, as best shown in FIG. 8. Therefore, the looped membrane 751 is interposed between the pressure channel 83 and the fluid channel 75, whereas the membrane valve 771 is interposed between the E-shaped pressure slot 861 and the micro-channel 77. The pressure-controlled layer 8 does not extend to the receptacle 74 formed in the fluid bearing layer 7 so that the receptacle 74 is uncovered.

The looped membrane 751 is inflatable and deflatable by the pressure in the sub-channels 831, 832, 833 so as to block or unblock the fluid channel 75 partially or completely. When the looped membrane 751 is deflated partially, one temperature zone of the fluid channel 75 is unblocked and the remaining temperature zones of the fluid channel 75 are blocked so that a fluid sample which is introduced into the fluid channel 75 may be moved to the unblocked temperature zone from the blocked temperature zones.

After assembly, the pressure inlet/outlet holes 851, 852, 853, and 863 are connected to a piping system (not shown) which is connected to a pneumatic device (not shown). A computerized control system may be used to control the pneumatic device to supply or withdraw gas to or from the pressure inlet/outlet holes 851, 852, 853 and 863.

Operation of the Chip Apparatus 500

A fluid sample for PCR is first placed in the receptacle 74. Prior to placement of the fluid sample, the pneumatic device is controlled to supply air to the pressure inlet/outlet holes 851, 852, 853 so that all of the sub-channels 831, 832 and 833 are pressurized through the micro-passages 841, 842, 843 and the looped membrane 751 is inflated completely to block the entire fluid channel 75. The E-shaped pressure slot 861 is also pressurized through the micro-passage 865 and the pressure inlet/outlet hole 863 to pressurize the membrane valve 771 so as to block the micro-channel 77 so that the fluid sample in the receptacle 74 does not flow into the micro-channel 77.

When the E-shaped pressure slot 861 is depressurized, the first, second and third sub-channels 831, 832, 833 are also depressurized so that the looped membrane 751 is deflated completely and the entire part of the fluid channel 75 is unblocked. The fluid sample is therefore permitted to flow into the fluid channel 75. At this stage, it is not needed to pressurize the three sub-channels 831, 832, 833 consecutively. After the fluid sample flows into the fluid channel 75, the micro-channel 77 is blocked by the membrane valve 771 inflated by the pressurized E-shaped pressure slot 861 so that the fluid sample is prevented from flowing back to the receptacle 74 through the micro-channel 77.

To thermally cycle the fluid sample, the second and third sub-channels 832, 833 are first pressurized to inflate the looped membrane 751 partially and to block the fluid channel 75 partially. That is to say, a portion of the looped membrane 751 immediately below the second and third sub-channels 832, 833 are inflated, and a portion of the fluid channel 75 therebelow are blocked. The fluid sample is therefore moved into and is collected in the unblocked portion of the fluid channel 75 immediately below the first sub-channel 831.

After the fluid sample is collected in the unblocked portion of the fluid channel 75 below the first sub-channel 831, the first, second and third heating devices 621, 622 and 623 in cooperation with and the first, second and third temperature sensors 631, 632 are set at three respective temperatures, namely, 95 deg, 53 deg, 72 deg. Therefore, three temperature zones are provided in the fluid channel 75. In particular, the temperature zone below the first sub-channel 831 has a temperature of 95 deg, the temperature zone below the second sub-channel 832 has a temperature of 53 deg, and the temperature zone below the third sub-channel 833 has 72 deg.

The fluid sample is thus collected in the temperature zone of 95 deg wherein the target DNA in the fluid sample is denatured to a single strand from a double helix structure. Afterwards, the second sub-channel 832 is depressurized, and the first sub-channel 831 is pressurized so that the fluid sample flows into the temperature zone of 53 deg below the second sub-channel 832, wherein a primer in the fluid sample is combined with the single strand DNA. Thereafter, the third sub-channel 833 is depressurized, and the second sub-channel 832 is pressurized so that the fluid sample flows into the temperature zone of 72 deg below the third sub-channel 833 wherein the single strand DNA is duplicated to a double helix. At this stage, one temperature cycle is completed for the fluid sample.

The aforesaid temperature cycle may be repeated until a predetermined number of cycles is completed. After completion of PCR, the E-shaped pressure slot 861 is depressurized and the first, second and third sub-channels 831, 832, 833 are pressurized to permit the fluid sample to flow into the receptacle 74 from the fluid channel 75.

While the pressure channel 83 and the fluid channel 75 are circular in this preferred embodiment, they may be provided with any other looped shapes, such as, an oval, or polygonal shape, according to the present invention.

As mentioned above, the chip device 500 according to the present invention is simple in either construction or operation and permits the fluid sample to flow rapidly from one temperature zone to another zone. The present invention eliminates the problem of time wastage encountered by the prior art due to the need to wait for the heating temperature to increase or decrease to a predetermined level.

In addition, the retention time of the fluid sample in each temperature zone of the fluid channel 75 may be altered or adjusted in the present invention by controlling the pressure level in each of the sub-channels 831, 832, 833. Therefore, the number of thermal cycles performed in the fluid channel 75 may also be altered or adjusted according to the different natures of fluid samples.

While the present invention has been described in connection with what is considered the most practical and preferred embodiment, it is understood that this invention is not limited to the disclosed embodiment but is intended to cover various arrangements included within the spirit and scope of the broadest interpretations and equivalent arrangements.

We claim:

1. A chip device for a thermally cycled reaction, comprising:
a looped pressure channel divided into a plurality of angularly oriented sub-channels;
a looped fluid channel substantially corresponding to and disposed below said looped pressure channel, said fluid channel having a plurality of temperature zones disposed below said sub-channels, respectively; and
a membrane disposed between said fluid channel and said pressure channel, said membrane being inflated or deflated by the pressure in said sub-channels so as to block or unblock said fluid channel partially or completely, wherein,
when said membrane is deflated partially, one of said temperature zones may be unblocked and the remaining ones of said temperature zones may be blocked so that a fluid sample introduced into said fluid channel may be moved to said one of said temperature zones from said remaining ones of said temperature zones.

2. The chip device of claim 1, wherein said sub-channels are discommunicated fluidly from each other.

3. The chip device of claim 2, further comprising a plurality of micro-passages connected respectively to said sub-channels, and a plurality of pressure inlet/outlet holes connected respectively to said micro-passages.

4. The chip device of claim 2, further comprising a fluid bearing layer, and a pressure controlled layer superimposed over said fluid bearing layer, said pressure controlled layer being formed with said pressure channel and said micro-passages, said fluid bearing layer being formed with said fluid channel, said membrane being a looped membrane formed in said fluid bearing layer between said pressure channel and said fluid channel.

5. The chip device of claim 4, wherein said fluid bearing layer includes top and bottom surfaces, said pressure controlled layer including top and bottom surfaces, said bottom surface of said pressure controlled layer being in contact with said top surface of said fluid bearing layer, said bottom surface of said fluid bearing layer being recessed to form said fluid channel which does not extends through said top surface of said fluid bearing layer, said bottom surface of said pressure controlled layer being recessed to form said pressure channel which does not extend through said top surface of said pressure controlled layer, a portion of said fluid bearing layer between said pressure channel and said fluid channel defining said looped membrane.

6. The chip device of claim 4, wherein said fluid bearing layer further includes a receptacle adapted to receive a fluid sample, and a micro-channel which is connected fluidly to said receptacle and said fluid channel.

7. The chip device of claim 6, further comprising a membrane valve to control said micro-channel of said fluid bearing layer, and a second pressure inlet/outlet hole provided in said pressure controlled layer and connected to said membrane valve.

8. The chip device of claim 4, wherein said pressure controlled layer and said fluid bearing layer are made of polydimethylsiloxane.

9. The chip device of claim 1, wherein said looped pressure channel is substantially annular.

10. The chip device of claim 9, wherein the number of said sub-channels is three, each of said sub-channels having a length substantially equal to one third of a circumferential length of said looped pressure channel.

11. The chip device of claim 4, further comprising a substrate disposed below said fluid bearing layer and formed with a plurality of heating devices to heat said fluid channel and to provide said fluid channel with said temperature zones.

12. The chip device of claim 11, wherein said substrate further includes a plurality of temperature sensors cooperating with said heating devices, respectively.

13. The chip device of claim 12, wherein said substrate further includes a plurality of pairs of conductor films, each pair of said conductor films being connected to one of said heating devices and said temperature sensors.

14. The chip device of claim 13, wherein each of said heating devices and said temperature sensors includes a film made of platinum, said conductor films being made of gold.

15. The chip device of claim 14, further comprising a partition plate disposed between said substrate and said fluid bearing layer.

16. The chip device of claim 15, wherein said partition plate is made of glass.
